(19) United States
(12) Patent Application Publication
(10) Pub. No.: US 2025/0128426 A1
(43) Pub. Date: Apr. 24, 2025

(54) TRANSPARENT AND FLEXIBLE ELECTRONIC SKIN AND METHOD FOR FABRICATING THE SAME

(71) Applicant: City University of Hong Kong, Hong Kong (HK)

(72) Inventors: Xinge YU, Hong Kong (HK); Jingkun ZHOU, Hong Kong (HK); Jian LI, Hong Kong (HK); Huiling JIA, Hong Kong (HK)

(21) Appl. No.: 18/492,808

(22) Filed: Oct. 24, 2023

(51) Int. Cl. B25J 13/08; A61L 27/60; B25J 13/00; B25J 19/00

(52) U.S. Cl. CPC B25J 13/088; B25J 13/006; B25J 13/081; B25J 13/087; B25J 19/005; A61L 27/60

(57) ABSTRACT

The present invention provides a transparent and flexible electronic skin with three-dimensional sensing capability. The electronic skin comprises: a substrate; an transparent and flexible electric field sensor disposed on the substrate; a control circuit disposed on the substrate; and a transparent and flexible protective layer encapsulating the electric field sensor and the control circuit. The electric field sensor includes a transmitter electrode; one or more receiver electrodes; and dielectric layer arranged between the transmitter electrode and receiver electrodes. The control circuit is connected to the electric field sensor and configured to: drive the transmitter electrode to transmit a reference signal to establish a quasi-static electrical near field; and process one or more measurement signals received by the one or more receiver electrodes respectively, each measurement signal is indicative of a distortion of the quasi-static electrical near field due to proximity of an object.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention generally relates to electronic skin technology. More specifically the present invention relates to a transparent and flexible electronic skin with three-dimensional sensing capability.

### BACKGROUND OF THE INVENTION

Three-dimensional sensing capability is crucial for human-machine interaction and machine perception in the three-dimensional world. With the popularity of topics such as metaverses and teleoperated robots in recent years, 3D spatial perception functions have become more and more indispensable. However, conventional skin electronics are limited to acquiring only two-dimensional spatial data through physical contact.

The field of 3D gesture recognition has seen rapid advancements in recent years. Some state-of-art wearable devices can detect finger movements in space, relying on inertial sensors, strain/stress sensors, ultrasound, or electrocardiogram signals. These devices often have limitations in recognizing a diverse range of gestures and dynamically tracking finger movements in space. Other strategies based on image analysis, magnetic fields, radar technology, or electrostatic principles come with their own challenges, such as complex processing algorithms, bulky device sizes, high power consumption, sensitivity to change in working environments and so on.

### SUMMARY OF THE INVENTION

One objective of the present invention is to provide a transparent and flexible electronic skin with three-dimensional sensing capability, simpler processing algorithm, compact size, low power consumption and adaptive to various working environments. Another objective of the present invention is to provide a method fabricating a transparent and flexible electronic skin which is suitable for mass production.

According to one aspect of the invention, a transparent and flexible electronic skin, which is inspired by active sensing system of Mormyroidea, is provided. The transparent and flexible electronic skin comprises: a transparent and flexible substrate; a transparent and flexible electric field sensor disposed on the substrate. The electric field sensor comprises: a transparent and flexible transmitter electrode; and one or more transparent and flexible receiver electrodes. The electronic skin further comprises a control circuit disposed on the substrate and connected to the electric field sensor and a transparent and flexible protective layer encapsulating the electric field sensor and the control circuit. The control module is configured to: drive the transmitter electrode to transmit a reference signal to establish a quasi-static electrical near field around the electric field sensor; and process one or more measurement signals received by the one or more receiver electrodes respectively, each measurement signal is indicative of a distortion of the quasi-static electrical near field due to proximity of an object to a corresponding receiver electrode.

According to another aspect of the invention, a method for fabricating a transparent and flexible electronic skin is provided. The method comprises: moulding a transparent and flexible material to form a patterned transparent and flexible substrate; dispensing a first conductive transparent gel on the patterned transparent and flexible substrate to form a transmitter electrode; forming a control circuit on the patterned transparent and flexible substrate; moulding the transparent and flexible material to form a patterned transparent and flexible dielectric layer; dispensing a second conductive transparent gel on the patterned transparent and flexible dielectric layer to form one or more receiver electrodes; attaching the dielectric layer on the substrate such that the transmitter electrode is spaced apart from the one or more receiver electrodes through the dielectric layer to form an electric filed sensor; and encapsulating the control circuit and the electric field sensor with the transparent and flexible material.

By employing bio-gel as electrode material and silicone polymers as substrates/dielectric layers, the electronic skin provided by the present invention has exceptional stretchability and light transmittance. This unique feature enables seamless integration with existing surfaces, such as screens, without imposing a significant reduction in brightness. Furthermore, the electric filed sensor in the provided electronic skin exhibit excellent performance in 3D sensing even when being submerged underwater. Through practical evaluation involving PCs, robots, and drones, the immense application potential of the present invention has been showcased in human-machine interaction (such as touch screens), virtual-reality space (such as metaverse) and robotics application (such as remote robot control) scenarios demanding three-dimensional manipulation and perception. Moreover, the feasibility of mass production through printing technology substantially reduces manufacturing costs.

The utilization of a bio-gel as an electrode not matches the performance of traditional metal electrodes but also enhances the device's stretchability, eliminating concerns about metal fatigue. The provided electronic skin is lightweight, transparent, wireless. Moreover, the overall performance of the electronic skin affixed to human skin can be significantly enhanced when using the human skin as a grounding system. Moreover, the provided electronic skin demonstrates superior underwater performance, indicating great potential for underwater applications.

### BRIEF DESCRIPTION OF THE DRAWINGS

Aspects of the present disclosure may be readily understood from the following detailed description with reference to the accompanying figures. The illustrations may not necessarily be drawn to scale. That is, the dimensions of the various features may be arbitrarily increased or reduced for clarity of discussion. There may be distinctions between the artistic renditions in the present disclosure and the actual apparatus due to manufacturing processes and tolerances. Common reference numerals may be used throughout the drawings and the detailed description to indicate the same or similar components.

FIG. 1 illustrates a photo of a transparent and flexible electronic skin according to one embodiment of the present invention.

FIGS. 2A and 2B illustrate schematic exploded views of the sensing and circuitry parts of the electronic skin respectively.

FIG. 3 shows an equivalent circuit model of the electric field sensor according to one embodiment of the present invention.

FIG. 4A shows a high frequency square wave voltage signal used by the control circuit to drive transmitter electrode; and FIG. 4B shows voltage signal received by receiver electrode.

FIG. 5 shows a plot of capacitance and received voltage with distance obtained by placing a target at various distances from the electric field sensor in the electronic skin.

FIG. 6 shows an exemplary electrode design according to one embodiment of the present invention.

FIG. 7 shows some critical parameters of the electronic skin according to one embodiment of the present invention.

FIG. 8 illustrate a functional block diagram of the control circuit 130 according to one embodiment of the present invention.

FIG. 9 shows a schematic flow of a method for fabricating a transparent and flexible electronic skin according to one embodiment of the present invention.

FIG. 10 shows light transmission of an electronic skin provided by the present invention.

FIG. 11 shows the change in capacitance during the stretching of the electronic skin for 25% from its original length.

FIG. 12 performance of the electronic skin after more than 8000 cycles of folds and unfolds.

FIG. 13 shows how the electronic skin is used to collect and process sensing signals due to proximity of a user's finger to obtain three-dimensional space coordinates of the finger within a predefined interactive space.

FIG. 14 shows the sensing signals received by the electronic skin and the finger movement trajectory calculated when using the finger to write five letters “CITYU” in the predefined interactive space.

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with various aspects of the present invention, a transparent and flexible electronic skin with three-dimensional sensing capability, which is inspired by active sensing system of Mormyroidea, is provided. Mormyroidea, a superfamily of weakly electric fish, are capable of using electric fields to detect prey hidden in the sediment underwater. These fishes generate a weak electric field through electric organs located in their tail, and then sense distortions in the electric field caused by the target through electroreceptors that are present throughout their body.

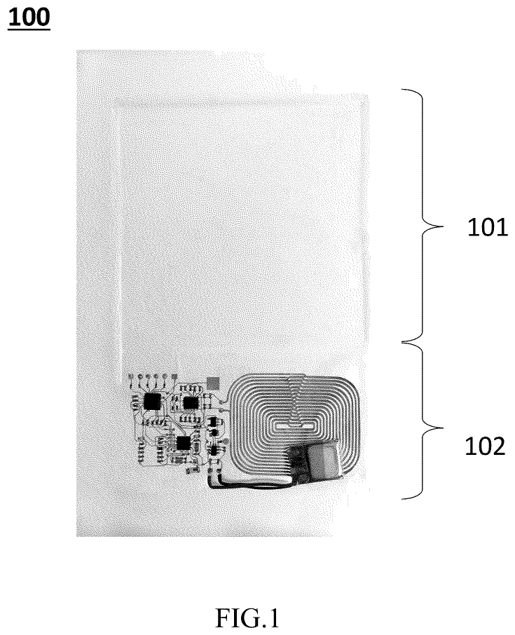

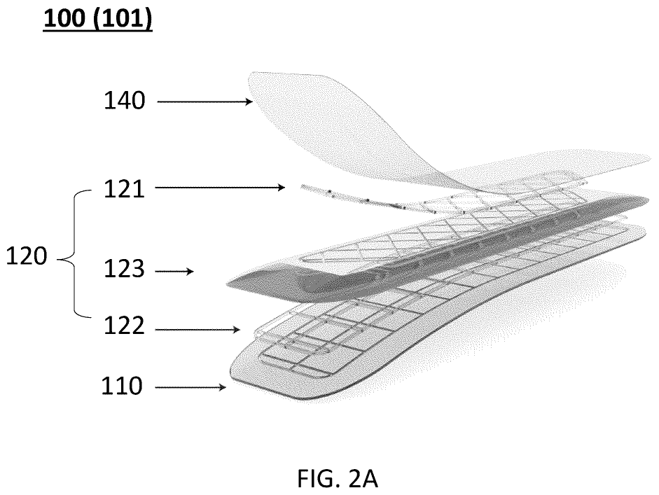

FIG. 1 illustrates a photo of a transparent and flexible electronic skin 100 according to one embodiment of the present invention. The electronic skin 100 may be divided into a sensing part 101 and a circuitry part 102. FIGS. 2A and 2B illustrate schematic exploded views of the sensing and circuitry parts respectively. As shown, the electronic skin 100 comprises a transparent and flexible substrate 110, a transparent and flexible electric filed sensor 120; a control circuit 130; and a transparent and flexible protective layer 140 for encapsulating the electric field sensor and the control circuit to protect the electric field sensor and the control circuit from external environmental factors, such as moisture or dust.

FIGS. 2A and 2B illustrate close-up views of the sensing parts 101 and circuitry part 102 of the electric field sensor 120 respectively. As shown, the electric field sensor 120 is disposed on the substrate 110 and covered by the protective layer 140. That is, the electric field sensor 120 is arranged between the substrate 110 and the protective layer 140. The electric field sensor 120 comprises a transparent and flexible transmitter electrode 121; one or more transparent and flexible receiver electrodes 122; and a transparent and flexible dielectric layer 123 arranged between the transmitter electrode and receiver electrodes.

Preferably, the substrate 110, dielectric layer 123 and protective layer 140 are made of silicone polymer, such as polydimethylsiloxane (PDMS). The electrodes may be made of conductive transparent gel, such as biogel containing NaCl and having an ionic conductivity of 34.3 S/m.

The control circuit 130 is disposed on the substrate 110 and connected to the electric field sensor 120. The control circuit 130 is configured to drive the transmitter electrode 121 to transmit a reference signal to establish a quasi-static electrical near field around the electric field sensor 120; and process one or more measurement signals received by the one or more receiver electrodes 122 respectively. Each measurement signal is indicative of a distortion of the quasi-static electrical near field (i.e., the reference signal) due to proximity of an object (e.g., a finger) to a corresponding receiver electrode.

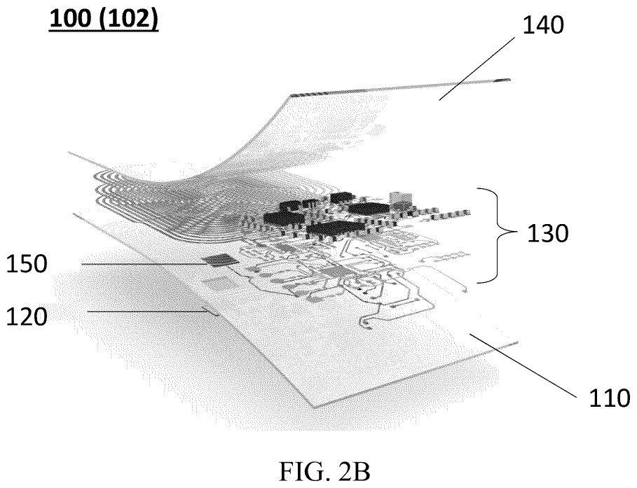

FIG. 3 shows an equivalent circuit model of the electric field sensor. The transmitter (Tx) and receiver (Rx) electrodes of the electric field sensor, along with the dielectric layer in the middle, form a flat capacitor with a capacitance value of C_{Sensor}. A variable capacitance is formed between the object (or target) to be detected or sensed (such as a human hand) and the receiver electrode, having a capacitance value denoted as C_{Hand}. Further, the capacitance formed by the two electrodes and the system ground are C_{RG} and C_{TG}, respectively. When using an external power supply, the system ground and earth ground are assumed to be equivalent. The capacitance between the target to be sensed and the earth ground is C_{HG}. When using battery power, there is an additional capacitance between the system ground and earth ground, denoted as C_{GND}. For sake of simplicity, C_{GND} and C_{HG} is omitted from the model. Given the driving signal V_{Tx} applied to the Tx electrode, the received signal V_{Rx} received by the Rx electrode can be expressed as:

$${V}_{R x} = {V}_{T x} \times \frac{{C}_{S e n s o r}}{{C}_{S e n s o r} + {C}_{R G} + {C}_{H a n d}} \qquad ( 1 )$$

This received signal V_{Rx} is dependent on the capacitance values C_{Sensor} and C_{Hand}, which can be estimated using the flat capacitor formula C=ε·ε₀·S/d, where ε is the relative dielectric constant, ε₀ is the vacuum dielectric constant, S is the effective area and d is the distance.

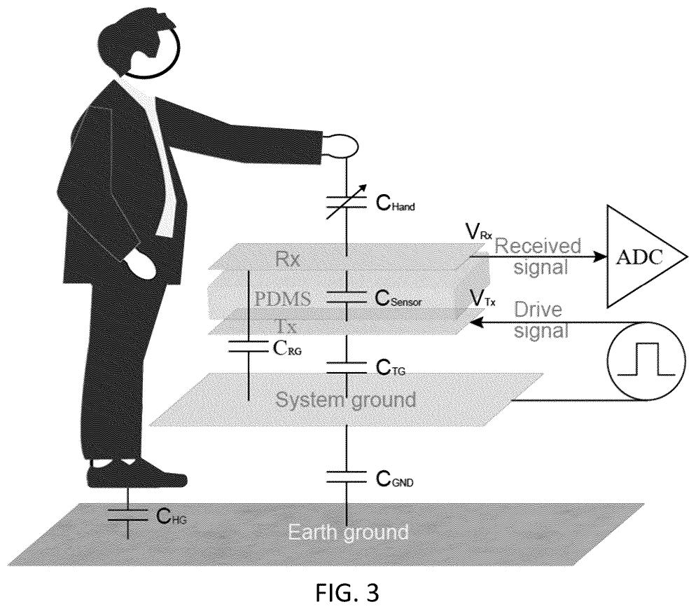

In some embodiments, the transmitter electrode may be driven by the control circuit with a high frequency square wave signal with amplitude of around 3V (as shown in FIG. 4A). The frequency of the square wave signal can be in a range of 44 k to 115 kHz.

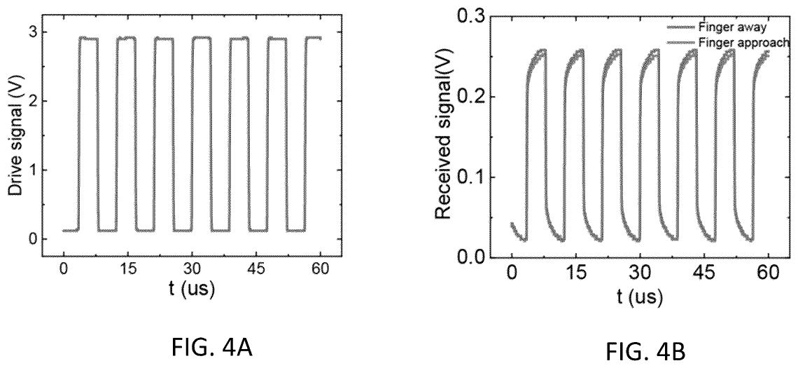

When the hand is approached to the sensor, C_{Hand} increases (S increases and d decreases in the flat capacitor formula), which leads to decrease of received signal V_{Rx} (as shown in FIG. 4B). The plot of capacitance and received voltage with distance (FIG. 5) can be obtained by placing the target at various distances from the sensor. According to Equation (1), when the variation range of C_{Hand} is fixed, to obtain a larger response, that is, make the variation of V_{Rx} as large as possible, the value of C_{Sensor} should be close to C_{RG}+C_{Hand}, and C_{RG}+C_{Hand} should be as small as possible.

The performance of the electronic skin may be affected by a variety of factors, such as the power source (USB or battery), the presence of a grounded shield beneath it, whether it is attached to the skin, and so on. When the device is powered by a battery, C_{HG} and C_{GND} cannot be ignored, resulting in a decrease in device performance. For example, when the electronic skin is directly attached to human skin, regardless of what types of power supply is used, its performance is significantly reduced due to the capacitance formed by the Rx electrode and the skin being added to the denominator of Equation (1) as a non-negligible term. To address this issue, the electronic skin 100 may further comprise a grounding pad 150 configured for electrically connecting a system ground of the electronic skin 100 to an earth ground.

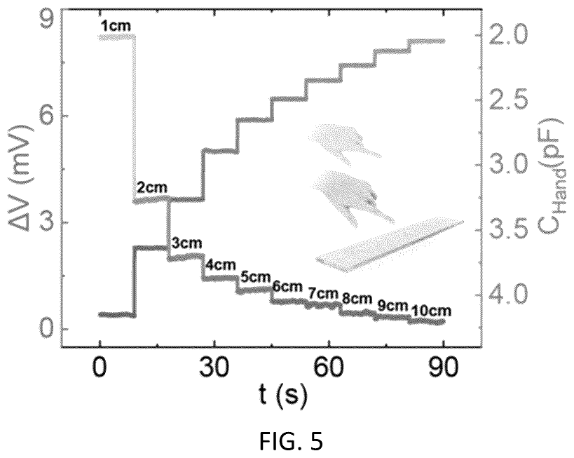

FIG. 6 illustrates an exemplary electrode design according to one embodiment of the present invention. As shown, each of the transmitter electrode and receiver electrodes may have a mesh pattern formed with conductive mesh lines. The receiver electrodes 122 may include four peripheral receiver electrodes 601 to 604 positioned at four peripheral regions of the electric filed sensor respectively; and a central receiver electrode 605 positioned at a central region of the electric field sensor.

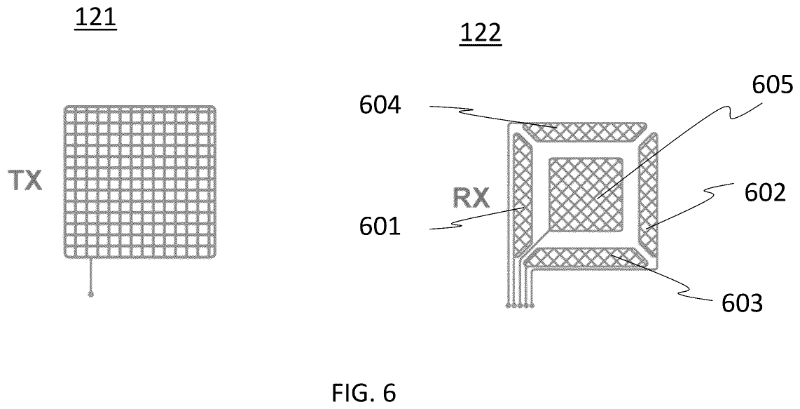

FIG. 7 shows some critical parameters of the electronic skin. Preferably, a ratio of the total conductive area of the transmitter electrode (denoted as A_{Tx_wire}, i.e., total area of conductive mesh lines) to the total electrode area of the transmitter electrode (denoted as A_{Tx}) is equal or less than 8.1%. Preferably, a ratio of the total conductive area of the receiver electrode (denoted as A_{Rx_wire}, i.e., total area of conductive mesh lines) to the total electrode area of the receiver electrode (denoted as A_{Rx}) is equal or less than 12.4%. The dielectric layer may have a thickness (denoted as d) in a range from 0.75 mm to 0.85 mm. Preferably, the thickness of the dielectric layer is equal to 0.8 mm.

Typically, the thickness of the substrate is 0.4 mm, the thickness of the dielectric layer is 0.6˜ 0.8 mm, the thickness of the biogel trace is 0.2 mm, and the width is 0.2 mm. The overall thickness of the electronic skin is ˜1 mm.

Moreover, an orientation of the conductive mesh lines of the transmitter electrode is substantially 45° different from an orientation of the conductive mesh lines of the receiver electrodes. In other words, the conductive mesh lines of the transmitter electrode are arranged to have an inclination angle substantially equal to 45° with respect to the conductive mesh lines of the receiver electrode.

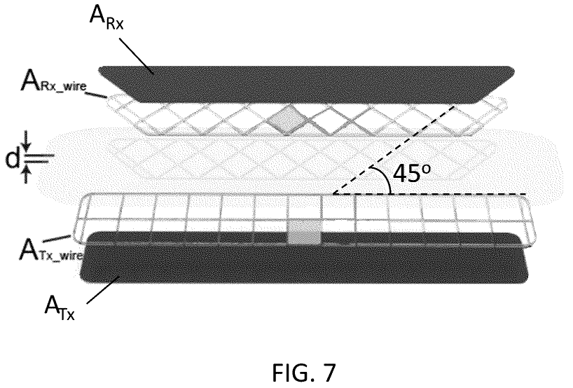

FIG. 8 illustrate a functional block diagram of the control circuit 130 according to one embodiment of the present invention. The control circuit may include a signal driver 801 configured to drive the transmitter (Tx) electrode to transmit the reference signal; and an analogy-to-digital converter 802 configured to convert the one or more measurement signals received from the receiver (Rx) electrodes to one or more digital signals respectively. The control circuit may further include a microprocessor 803 configured to: process the one or more digital signals to calculate one or more distances of the object relative to the one or more receiver electrodes respectively; and analyze the one or more calculated distances to determine a position/gesture data of the object. The control circuit may further comprise a wireless communication unit 804 configured to send the position/gesture data to a remote device 805, such as a mobile phone. In some embodiments, the wireless communication unit 804 is a Bluetooth Low Energy (BLE) communication module.

In some embodiments, distance of the object relative to each of the one or more receiver electrode is calculated by the processor 803 based on a least squares multilateration algorithm.

Preferably, the electronic skin may further comprise a lithium-ion battery 806; a capacitive touch switch 807 for switching connection of the battery 806 to the control unit; and a low-dropout regulator (LDO) 808 for regulating a voltage of the battery to a fixed, direct-current voltage to the control circuit.

The electronic skin may further comprise a wireless charger receiver 809 configured to receive electromagnetic energy from a wireless charger transmitter 810 and convert the received electromagnetic energy into DC voltage to charge the lithium-ion battery 806.

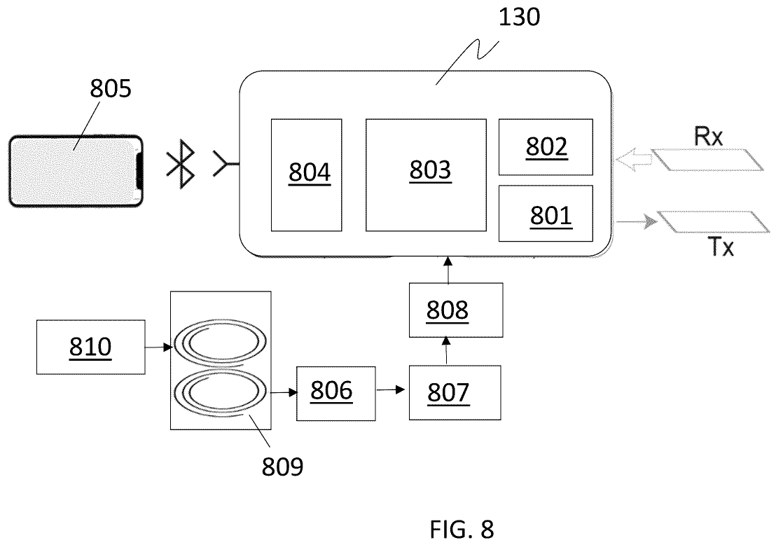

FIG. 9 shows a schematic flow of a method for fabricating a transparent and flexible electronic skin according to one embodiment of the present invention. As shown, the method includes:
S901: moulding a transparent and flexible material with a mold to form a patterned transparent and flexible substrate.
S902: dispensing a conductive transparent gel on the patterned transparent and flexible substrate to form a transmitter electrode;
S903: forming a control circuit on the patterned transparent and flexible substrate;
S904: moulding the transparent and flexible material with a mold to form a patterned transparent and flexible dielectric layer;
S905: dispensing the conductive transparent gel on the patterned transparent and flexible dielectric layer to form one or more receiver electrodes;
S906: attaching the dielectric layer on the substrate such that the transmitter electrode is spaced apart from the one or more receiver electrodes through the dielectric layer to form an electric filed sensor; and
S907: encapsulating the control circuit and the electric field sensor with the transparent and flexible material.

In some embodiments, a light-curing 3D printer (Creality Halot Box, Shenzhen Creality 3D Technology Co., Ltd.) with resin (Polyacrylate) may be used to manufacture the molds with patterns for forming the patterned transparent and flexible substrate/dielectric layer.

In some embodiments, a silicone polymer, such as PDMS (Sylgard 184, Dow Corning Corporation, 20:1) may be used the transparent and flexible material. In step S901/S904, the PDMS is poured into the mold and cured at 100° C. for 30 minutes to obtain the patterned transparent and flexible substrate/dielectric layer. In step S907, the PDMS is used to encapsulate the control circuit and the electric field sensor and left to cure at room temperature overnight.

In some embodiments, the conductive transparent gel may be prepared by: dissolving sodium chloride, citric acid, sodium citrate and glycerol in deionized water to form a first solution; and dissolving gelatin powder (300-g Bloom) in the first solution to form the conductive transparent gel.

In one embodiment, the weight ratio of sodium chloride, citric acid, sodium citrate, glycerol and deionized water in the first solution is substantially equal to 10:1:10:60:80. The weight ratio of the first solution and the gelatin power is substantially equal to 4:1. For instance, the conductive transparent gel may be prepared by dissolve 1 g of sodium chloride, 0.1 g of citric acid, 1 g of sodium citrate and 6 g of glycerol in 8 ml of deionized water, mix on a stirrer for 20 minutes, after which 4 g of gelatin powder should be added and heated in an oven at 75° C. for 4 hours to fully dissolve the gelatin. After cooling, the liquid will transform into a gel due to the physical cross-linking of the gelatin chains and the chemical cross-linking of the citrate ions with the gelatin chains. When using the biogel, just heat it to revert it to liquid state, which will allow it to be extruded from the needle while dispensing, and as the temperature drops to room temperature, the gel will revert back to tis gel-like state and adhere to the substrate.

In steps S902/S905, the biogel is placed in a barrel. The biogel is then heated to 50° C. and injected/dispensed into the electrode pattern channels on the PDMS substrate/dielectric layer through a printing or dispensing process to form the transmitter/receiver electrodes.

In some embodiments, the control circuit is formed on the patterned transparent and flexible substrate by: transfer printing a conductive trace pattern from a donor substrate to the patterned transparent and flexible substrate; and attaching electronic components on the patterned transparent and flexible substrate to form the control circuit.

### Performance Evaluation

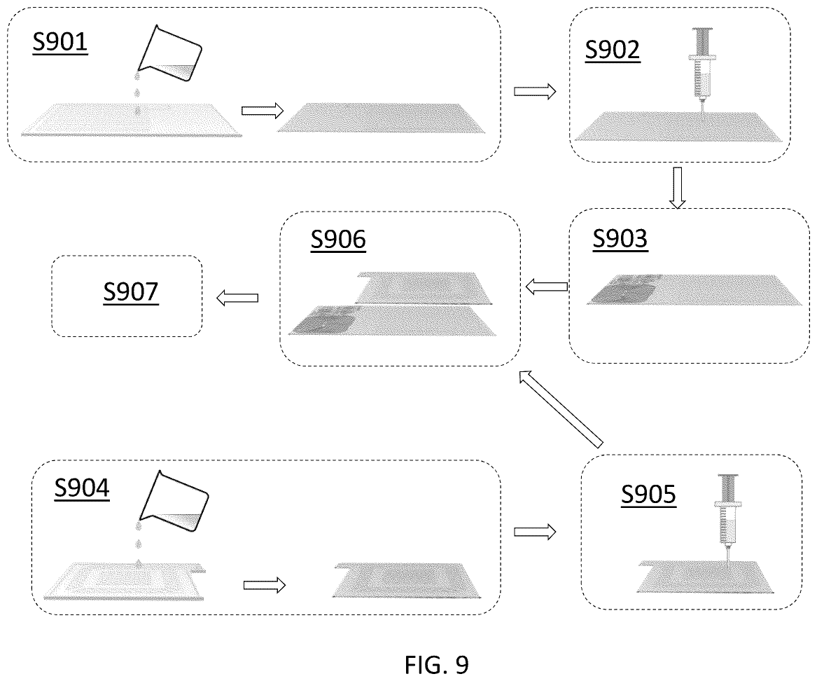

FIG. 10 shows light transmission of an electronic skin provided by the present invention. As shown, the electronic skin demonstrates a light transmission of more than 80% in the 410 nm-1000 nm range. Such high level of transparency allows the electronic skin to be used as a transparent 3D sensing and gesture recognition layer. For example, the electronic skin can be directly applied on a display or an infrared sensor, without blocking any line of sight. Furthermore, the transparency of the electronic skin allows for its integration, as a virtually invisible surface layer, into existing devices without changing the aesthetic of the device.

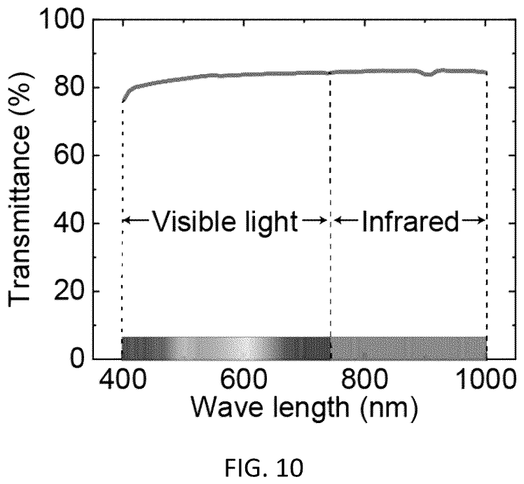

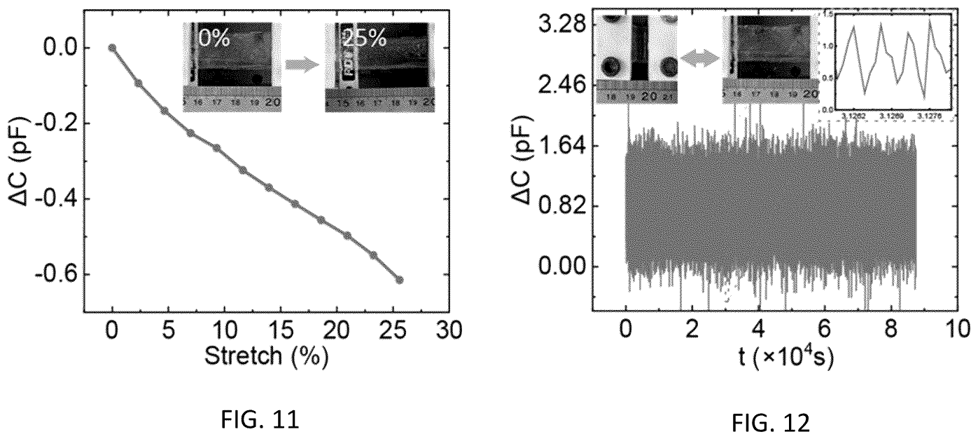

FIG. 11 shows the change in capacitance during the stretching of an electronic skin provided by the present invention from its original length to 25%. FIG. 12 shows that there was no significant change in device performance after more than 8000 folds and unfolds. Because conductive biogel is used to make the electrodes, there is no fatigue effect like that of metals which can experience wear and tear over time. This means that the electronic skin can be stretched and bent repeatedly without experiencing any damage.

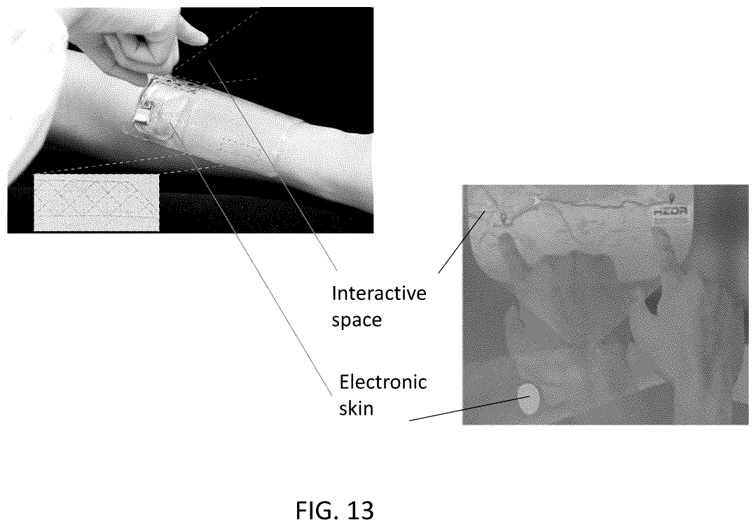

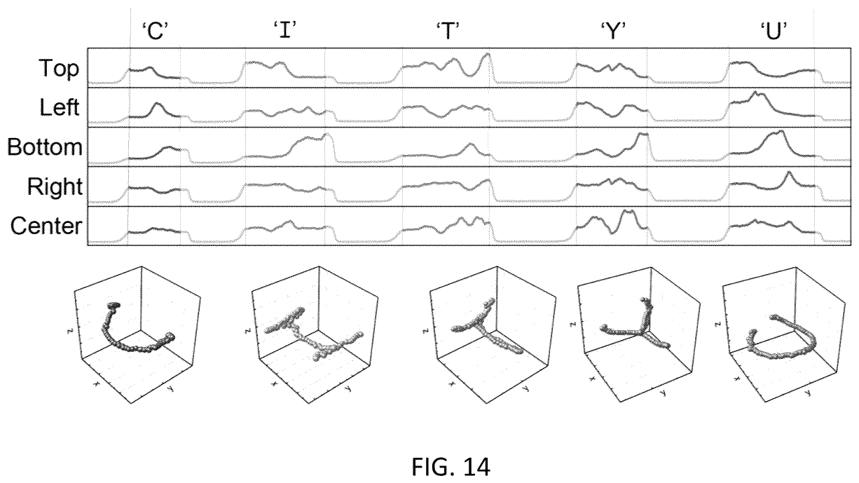

The electronic skin provided by the present invention can be used for gesture recognition and finger position tracking. As shown in FIG. 13, the electronic skin may be used to collect and process sensing signals due to proximity of a user's finger to obtain three-dimensional space coordinates of the finger within a predefined interactive space. FIG. 14 shows the sensing signals received by the electronic skin and the finger movement trajectory calculated when using a finger to write five letters “CITYU” in the predefined interactive space. The electronic skin is very suitable for 3D modeling, AR/VR, wearable devices and other applications that need to operate in three-dimensional space.

For instance, the electronic skin can also be used to empower a robot or a drone to perceive its surroundings more effectively and provide a more comprehensive solution for 3D object detection and distance measurement. For instance, a robot arm can use the electronic skin to judge the distance between a target or whether it has grasped the target. A drone can use the electronic skin to determine whether there are obstacles such as an electric cable on its path.

In underwater applications, the electronic skin can serve as a gesture recognition sensor in murky water to help divers to communicate effectively when their sight is blocked. This technology can also be used to monitor sea life and study collective animal behavior in the wild. This technology can help us to understand and protect marine ecosystems and the species that inhabit them.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A transparent and flexible electronic skin with three-dimensional sensing capability, comprising:
a transparent and flexible substrate;
a transparent and flexible electric field sensor disposed on the substrate and comprising:
a transparent and flexible transmitter electrode; and
one or more transparent and flexible receiver electrodes;
a control circuit disposed on the substrate and connected to the electric field sensor; and
a transparent and flexible protective layer encapsulating the electric field sensor and the control circuit; and
wherein the control circuit is configured to:
drive the transmitter electrode to transmit a reference signal to establish a quasi-static electrical near field around the electric field sensor; and
process one or more measurement signals received by the one or more receiver electrodes respectively, each measurement signal is indicative of a distortion of the quasi-static electrical near field due to proximity of an object to a corresponding receiver electrode.

2. The transparent and flexible electronic skin according to claim 1, wherein the one or more receiver electrodes include:
a central receiver electrode positioned at a central region of the electric field sensor; and
four peripheral receiver electrodes positioned at four peripheral regions of the electric filed sensor respectively.

3. The transparent and flexible electronic skin according to claim 1, wherein the transparent and flexible substrate are made of polydimethylsiloxane.

4. The transparent and flexible electronic skin according to claim 1, wherein the transparent and flexible protective layer are made of polydimethylsiloxane.

5. The transparent and flexible electronic skin according to claim 1, wherein the transparent and flexible dielectric layer is made of polydimethylsiloxane.

6. The transparent and flexible electronic skin according to claim 1, wherein the transparent and flexible transmitter and receiver electrodes are made of a conductive transparent gel.

7. The transparent and flexible electronic skin according to claim 6, wherein the conductive transparent gel contains sodium ions.

8. The transparent and flexible electronic skin according to claim 1, wherein the control circuit comprises:
a signal driver configured to drive the transmitter electrode to transmit the reference signal; and
an analogy-to-digital converter configured to convert the one or more measurement signals to one or more digital signals respectively.

9. The transparent and flexible electronic skin according to claim 8, wherein the control circuit further comprises a microprocessor configured to:
process the one or more digital signals to calculate one or more distances of the object relative to the one or more receiver electrodes respectively; and
analyse the one or more calculated distances to determine a position/gesture data of the object.

10. The transparent and flexible electronic skin according to claim 9, the control circuit further comprises a wireless communication unit configured to send the position/gesture data to a remote device.

11. The transparent and flexible electronic skin according to claim 10, wherein the distance of the object relative to each of the one or more receiver electrode is calculated by the processor based on a least squares multilateration algorithm.

12. The transparent and flexible electronic skin according to claim 1, further comprising
a lithium-ion battery for supplying power to the control unit; and
a wireless charging unit configured to receive electromagnetic energy and convert the received electromagnetic energy into DC voltage to charge the lithium-ion battery.

13. A method for fabricating a transparent and flexible electronic skin, comprising:
moulding a transparent and flexible material to form a patterned transparent and flexible substrate;
dispensing a first conductive transparent gel on the patterned transparent and flexible substrate to form a transmitter electrode;
forming a control circuit on the patterned transparent and flexible substrate;
moulding the transparent and flexible material to form a patterned transparent and flexible dielectric layer;
dispensing a second conductive transparent gel on the patterned transparent and flexible dielectric layer to form one or more receiver electrodes;
attaching the dielectric layer on the substrate such that the transmitter electrode is spaced apart from the one or more receiver electrodes through the dielectric layer to form an electric filed sensor; and
encapsulating the control circuit and the electric field sensor with the transparent and flexible material.

14. The method according to claim 13, wherein the control circuit is formed on the patterned transparent and flexible substrate by:
transfer printing a conductive trace pattern from a donor substrate to the patterned transparent and flexible substrate; and
attaching electronic components on the patterned transparent and flexible substrate to form the control circuit.

15. The method according to claim 13, wherein each of the first and second the conductive transparent gels is prepared by:
dissolving sodium chloride, citric acid, sodium citrate and glycerol in deionized water to form a first solution; and
dissolving gelatin powder in the first solution to form the conductive transparent gel.
